(19) United States
(12) Patent Application Publication
(10) Pub. No.: US 2005/0241075 A1
(43) Pub. Date: Nov. 3, 2005

(54) Composition for permanently reshaping the hair comprising at least one reducing agent and at least one photo-oxidizing agent complexed with at least one metal

(76) Inventors: Aude Livoreil, Paris (FR); Gabin Vic, Venette (FR); Henri Samain, Bievres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/111,709

(22) Filed: Apr. 22, 2005

Related U.S. Application Data
(60) Provisional application No. 60/571,917, filed on May 18, 2004.

(30) Foreign Application Priority Data
Apr. 22, 2004 (FR) 04 50764

(51) Int. Cl. A61K 7/13

(52) U.S. Cl. 8/405

(57) ABSTRACT

The present disclosure relates to a cosmetic reducing composition for permanently reshaping keratinous fibers, such as the hair, comprising, in a cosmetically acceptable medium, at least one reducing agent and at least one photo-oxidizing agent complexed with at least one metal. The present disclosure also relates to a process for permanently reshaping keratinous fibers with the compositions disclosed, and a multi-compartment kit containing such compositions for permanantly reshaping keratinous fibers.

This application claims benefit of U.S. Provisional Application No. 60/571,917, filed May 18, 2004, and French Patent Application No. 04/50764, filed Apr. 22, 2004, both of which are herein incorporated by reference.

The present disclosure relates to a cosmetic reducing composition, which can be used in a process to permanently reshape the hair, comprising at least one reducing agent and at least one photo-oxidizing agent. The present disclosure also relates to a method for permanently reshaping the hair using at least one composition as disclosed herein.

The most common method to obtain a permanent reshaping of the hair consists, in a first step, in opening the keratin disulfide bonds (cystine) with a composition comprising a reducing agent, and for instance, after rinsing the hair, in reforming, in a second step, the disulfide bonds by applying to the hair that has been straightened or placed beforehand under tension with suitable means, such as rollers, an oxidizing composition also called fixing solution, so as to give the desired form to the hair. This method can enable, the waving of the hair, the uncurling, the crimping or the straightening of the hair.

The common techniques to reshape the hair, such as the compositions to permanently reshape or straighten the hair that are based on thiol-group-comprising products may lead to good shaping results, but they can frequently provide the hair with an unpleasant odor.

To compensate for this drawback, solutions most commonly used in prior art compositions typically use a fragrance to mask bad odors. Nevertheless, such solutions are not always satisfactory as they do not remove these bad odors.

Alternative solutions have been proposed. Systems have been proposed to physically entrap odors, for instance, zeolite-based systems. U.S. Pat. No. 5,184,630 suggests, for example, adding a zeolite to a cosmetic composition so as to reduce bad odors. However, the odor reducing effect of these physically entrapping systems may not be sufficient.

It has also been suggested to carry out a manganese catalysis rather than use the peroxide in the fixing step of methods for permanently reshaping the hair.

However, if successive permanent waving then hair coloring operations follow each other, these compositions can induce a perturbation in the color uptake that can cause the resulting color shade to be too loud or strong.

Therefore, there is a need for new cosmetic compositions for use in a process to permanently reshape the hair, which can have a great efficiency to reduce bad odors and without the drawbacks of prior art, such as without inducing any perturbation in the color uptake during successive permanent waving/hair coloring operations.

The inventors have discovered, surprisingly, that a composition comprising at least one photo-oxidizing agent complexed with at least one metal can be used in a process to permanently reshape keratinous fibers and may result in a drastic reduction of bad odors without inducing any perturbation in the color uptake during successive permanent waving/coloring operations, and may provide at the same time an intensive and long lasting hold curling.

Photo-oxidizing agents catalyze decomposition of small-sized thiol group molecules through the atmospheric oxygen and can efficiently reduce unpleasant odors.

As used herein, the term “photo-oxidizing agents” is understood to mean molecules or complexes that may trigger an oxidizing reaction upon illumination, most often in the visible light range ranging from 400 nm to 800 nm. Exposing a photo-oxidizing agent to light brings it to a high energetic state. The so-called excited-photo-oxidizing agent then transfers its own energy to oxygen molecules that exist in the medium, thereby converting them into activated oxygen. This oxygen form is highly reactive and may easily oxidize other chemical species that are present in the medium, for example, some sulfur-containing species.

Accordingly, one aspect of the present disclosure is a cosmetic reducing composition for permanently reshaping keratinous fibers, for instance, the hair, comprising in a cosmetically acceptable medium at least one reducing agent and at least one photo-oxidizing agent complexed with at least one metal.

The at least one metal can be chosen from transition metals. Non-limiting mention of transition metals that may be used include Ti, V, Cr, Mn, Fe, Co, Ni, Cu, Zn, Mo, Ru, Rh, Pd and Ag. For example, the at least one metal may be chosen from Cu, Zn, Ni, Fe, Co and Mn, such as Fe and Mn.

Further non-limiting mention of suitable metals that may be used include those chosen from Si, Al, Ga, Ge and Sn.

The metal for the photo-oxidizing agent complexing generally can be provided as a metal salt, a metal oxide or a metal complex with at least one ligand.

According to one embodiment of the present disclosure, the at least one metal is a metal salt chosen from those of formula (I):
M(T)ₙ (I)
wherein

M is a metal chosen from Ti, V, Cr, Mn, Fe, Co, Ni, Cu, Zn, Mo, Ru, Rh, Pd and Ag metals,

T is an inorganic anion chosen from sulfate, sulfonate, phosphate, phosphonate, chloride, bromide and nitrate ions, or an organic anion chosen from lactate, citrate and acetate ions; and

n is an integer ranging from 1 to 8, such as 2 or 3.

According to another embodiment of the present disclosure, the at least one metal is a metal oxide chosen from those of formula (II):
M_{m′}(O)ₘ (II)
wherein

M is a metal chosen from Ti, V, Cr, Mn, Fe, Co, Ni, Cu, Zn, Mo, Ru, Rh, Pd and Ag metals,

m′ is an integer ranging from 1 to 5, and m is an integer ranging from 1 to 8. For example, m may be 2 or 3.

According to yet another embodiment of the present disclosure, the at least one metal is a metal complex with at least one ligand, wherein the complex is chosen from those of formula (III):
M(L)ₚ (III)
wherein

M is a metal chosen from Ti, V, Cr, Mn, Fe, Co, Ni, Cu, Zn, Mo, Ru, Rh, Pd and Ag metals,

L is an organic ligand chosen from saturated and unsaturated, cyclic and acyclic compounds, comprising carboxylate and/or primary, secondary or tertiary amine and/or hydroxy groups; or an inorganic ligand chosen from NH₃ and NO; and

p is an integer ranging from 1 to 10, such as 2 or 3.

L can be an organic ligand, by way of non-limiting example, chosen from pyridine, pyridazine, pyrimidine, pyrazine, imidazole, pyrazole, triazole, 2,2′-bispyridylamine, tris-(2-pyridylmethyl)amine, 1,4,7-triazaclyclononane, 1,4,7-trimethyl-1,4,7-triazaclyclononane, 1,5,9-trimethyl-1,5,9-triazacyclododecane, ((bis-(1-methylimidazol-2-yl)-methyl))-(2-pyridylmethyl)-amine, N,N′-((bis-(1-methylimidazol-2-yl)-methyl))-ethylenediamine, N-bis-(2-benzimidazolyl methyl)-aminoethanol, 2,6-bis-(bis-(2-benzimidazolylmethyl)aminomethyl)-4-methylphenol, N,N,N′,N′-tetrakis-(2-benzimidazolylmethyl)-2-hydroxy-1,3-diaminopropane, 2,6-bis(bis-(2-pyridylmethyl)aminomethyl)-4-methylphenol, 1,3-bis(bis-(2-benzimidazolylmethyl)aminomethyl)-benzol, sorbitol, mannitol, erythritol, adonitol, inositol, lactose and optionally salts thereof.

By way of further non-limiting example, the organic ligand may also be chosen from 1,4,8,11-tetraazacyclotetradecane, 1,8-diazabicyclo[5.4.0]undec-7-ene, ethylenediamine, nitrilotriacetic acid, tris(aminoethyl)amine, tris(aminomethyl)methane, 1,3,5-triaminocyclohexane, dicarboxylic 2-6-pyridine, 2-6-pyridine-dicarboxylic acid, 2,2′,6′,2″-terpyridine, 1,10-phenanthroline, tris(2-pyridylmethyl)amine, tris(2-pyridylethyl)amine and N-carboxymethyl-N-(2-pyridylmethyl)glycine.

A previously complexed photo-oxidizing agent, for instance, can also be directly used in the composition according to the present disclosure.

As explained above, the composition according to the present disclosure comprises at least one photo-oxidizing agent complexed with at least one metal. The metal in the complexed photo-oxidizing agent has an oxidation number of greater than 0.

The at least one photo-oxidizing agent can be chosen, for example, from:
porphyrines,
porphyrine derivatives chosen from chlorine, pheophorbide, pyreophorbide and benzoporphyrine derivatives,
naphtalocyanines,
phtalocyanines; and
cyanocobalamine.

According to one embodiment of the present disclosure, the at least one phtalocyanine complexed with the at least one metal can be chosen from compounds of formula (IV): 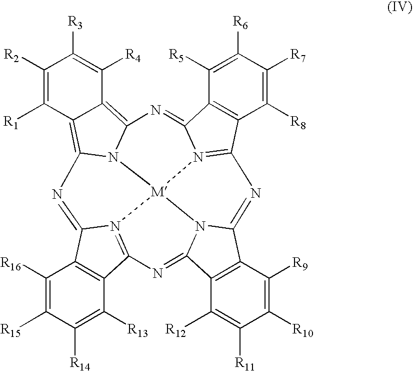
wherein

M′ is a metal chosen from Ti, V, Cr, Mn, Fe, Co, Ni, Cu, Zn, Mo, Ru, Rh, Pd and Ag metal, such as Cu, Zn, Ni, Fe, Co and Mn, for instance, Fe and Mn, wherein the M′ metal has an oxidation number of greater than 0 and being optionally flanked by at least one organic or inorganic anion such as defined above and/or by at least one ligand such as defined above,

R₁ to R₁₆, which may be identical or different, are chosen from:
hydrogen,
hydroxy, amino, mono and dialkylamino, mono and dihydroxyalkylamino, alkoxy, halogeno, and cyano moieties,
saturated and unsaturated, linear and branched hydrocarbon chains comprising from 1 to 50 carbon atoms, optionally interrupted by at least one entity chosen from heteroatoms, such as S, O, N, and P atoms; optionally substituted, aromatic and heteroaromatic rings, such as hydroxy or alkylhydroxy groups; and carbonyl groups; the chains being optionally substituted by at least one group chosen from hydroxy, amino, alkoxy, halogeno, cyano, mono and dialkylamino, and mono and dihydroxyalkylamino groups,
radicals bearing solubilizing ionic groups,
radicals bearing at least one reactive group that can graft phtalocyanine onto a solid carrier or onto a soluble or insoluble polymer.

Solubilizing ionic groups are typically selected from carboxylate, sulfonate, trialkylammonium, trialkylphosphonium, triarylphosphonium, alkylpyridinium and arylpyridinium groups, alkyl residues being C₁-C₁₈ and aryl residues C₆-C₁₂.

Suitable phtalocyanines for use according to the present disclosure, include, by way of non-limiting example, copper II phtalocyanine commercially marketed by Aldrich under reference number 61218, manganese II phtalocyanine commercially marketed by Aldrich under reference number 37,955-7 and iron III phtalocyanine commercially marketed by Fluka under reference number 44968.

The at least one photo-oxidizing agent can be soluble in the composition or be dispersed into the composition.

According to still another embodiment, the at least one photo-oxidizing agent is fixed to a solid carrier, so as to limit the penetration into the keratinous fibers. In such an embodiment, the at least one photo-oxidizing agent may thus be adsorbed or grafted onto a solid carrier, typically by means of a covalent bond on this carrier. The solid carrier is can be chosen from silica particles, titanium oxide particles, polymer particles and metal particles. Suitable polymer particles for use as a carrier include polypropylene, polyurethane or styrene-divinylbenzene particles.

Phtalocyanine grafting and/or adsorption methods onto various carriers are inter alia described in the following publications:
“Synthesis and catalytic properties of a novel phthalocyanine covalently grafted onto silica,” S. Mangematin, A B Sorokin, Journal of Porphyrins and Phthalocyanines, 2001, 5, p 674-680;
“Fabrication of deodorizing fabric by grafting metal phthalocyanine derivative onto non woven polypropylene fabric,” Journal of Applied Polymer Science, 2001, 82(4), p 839-846;
“Fabrication of gas sensing films based on nanoparticles grafted with electronically or chemically interacting organic molecules,” WO 2000014520, CEA
“In vivo comparison of blood compatibility of iron-phthalocyanine grafted polyurethanes,” Advances in Biomaterials, 1980,1, p 519-527;
“Macroporous styrene-divinylbenzene copolymers as carriers for polyvinylamine-cobalt phthalocyanine oxidation catalysts,” Angewandte Makromolekulare Chemie, 1980, 89, p 201-219.

Fixing the at least one photo-oxidizing agent can also be conducted by entrapment. The at least one photo-oxidizing agent can be entrapped within a porous material, for instance, selected from polymer particles such as ORGASOL type-polyamides, three-dimensional lattices resulting from alkoxy silane molecules condensation, and zeolites. Three-dimensional lattices that can be used include, by way of non-limiting example those described in the publication: “Encapsulation of iron phthalocyanine in sol-gel materials,” A. B. Sorokin, P. Buisson, A. C. pierre, Microporous and Mesoporous materials, 2001, 46, p 87-98.

The at least one photo-oxidizing agent can be present in an amount ranging from 0.1% to 20%, such as from 1% to 10% by weight, relative to the total weight of the composition.

As disclosed above, the composition according to the present disclosure comprises at least one reducing agent in addition to at least one photo-oxidizing agent complexed with at least one metal atom.

The at least one reducing agent can be chosen from those of formula (VI):
(H)_{q}X(R)ᵣ (VI)
wherein X is chosen from P and S atoms, and SO₂ groups, q is 0 or 1, r is 1 or 2 or 3, and R is chosen from linear and branched, saturated and unsaturated (C₁-C₂₀)hydrocarbon radicals, optionally interrupted by one heteroatom, and optionally substituted with at least one group chosen from hydroxy, halogeno, amine, carboxy, ((C₁-C₃₀) alkoxy)carbonyl, amido, ((C₁-C₃₀)alkyl)amino carbonyl, ((C₁-C₃₀)acyl) amino, mono and di((C₁-C₃₀)alkyl)amino, mono and dihydroxy ((C₁-C₃₀)alkyl)amino groups, the salts thereof combined with a base.

Suitable reducing agents include, by way of non-limiting example, thioglycolic acid and thiolactic acid and ester and amide derivatives thereof, such as glycerol monothioglycolate; cysteamine and its (C₁-C₄) acylated derivatives such as N-acetyl-cysteamine or N-propionyl-cysteamine; cysteine; N-acetyl-cysteine; thiomalic acid; pantheteine; 2,3-dimercaptosuccinic acid; N-(mercaptoalkyl)-ω-hydroxyalkylamides such as those described in European Patent Application No. EP-A-354 835; N-mono and N,N-dialkylmercapto-4-butyramides such as those described in European Patent Application No. EP-A-368 763; aminomercaptoalkylamides, such as those described in European Patent Application No. EP-A-432 000; N-(mercaptoalkyl)succinamic acid derivatives and N-(mercaptoalkyl)succinimide derivatives such as those described in European Patent Application No. EP-A-465 342; alkylamino mercaptoalkylamides such as those described in European Patent Application No. EP-A-514 282; an azeotropic mixture of 2-hydroxypropyl thioglycolate and (2-hydroxy-1-methyl)ethyl thioglycolate described in French Patent Application No. FR-A-2 679 448; mercaptoalkylaminoamides such as those described in French Patent Application No. FR-A-2 692 481; N-mercaptoalkylalkanediamides described in European Patent Application No. EP-A-653 202, as well as formamidine sulfinic acid derivatives. If desired, these compounds can also be used as salts.

The at least one reducing agent can be present in an amount ranging from 0.05% to 30%, such as from 1% to 20% by weight, relative to the total weight of the composition.

The pH value of the composition according to the present disclosure can range from 4 to 11, for instance, from 6 to 10.

The pH value can be balanced by means of an alkaline agent such as, for example, ammonia, monoethanolamine, diethanolamine, triethanolamine, 1,3-propanediamine, alkaline or ammonium carbonate or bicarbonate, organic carbonate such as guanidine carbonate, alkaline hydroxide; or by means of an acidifying agent such as for example hydrochloric acid, acetic acid, lactic acid, oxalic acid or boric acid; or by means of usual buffers as well, such as for example borate, phosphate or TRIS-buffers (tris(hydroxymethyl) amino methane-based buffers).

In one embodiment of the present disclosure, the reducing composition as disclosed herein also comprises at least one surfactant chosen from non ionic, anionic, cationic and amphoteric surfactants. Non-limiting examples of surfactants that may be used include alkyl sulfates, alkyl benzene sulfates, alkyl ether sulfates, alkyl sulfonates, quaternary ammonium salts, alkyl betaines, oxyethylene alkyl phenols, fatty acid alkanolamides, oxyethylene fatty acid esters, and other non ionic hydroxypropylether surfactants.

When the reducing composition as disclosed herein comprises at least one surfactant, the at least one surfactant can be present in an amount less than or equal to 30% by weight, such as ranging from 0.5% to 10% by weight, relative to the total weight of the reducing composition.

In order to improve the cosmetic properties of the hair, or to reduce or prevent the damaging effects, the reducing composition may also comprise at least one treating agent chosen from those of cationic, anionic, non ionic and amphoteric nature.

Among the treating agents that may be used, non-limiting mention may be made of those described in French Patent Nos. FR 2 598 613 and FR 2 470 596. Additional non-limiting mention may be made of volatile and/or non volatile, linear and/or cyclic silicones, polydimethylsiloxanes, quaternized polyorganosiloxanes, such as those described in French Patent Application No. FR 2 535 730, polyorganosiloxanes with aminoalkyl groups modified by alkoxycarbonylalkyl moieties such as those described in U.S. Pat. No. 4,749,732, polyorganosiloxanes such as polydimethylsiloxane-polyoxyalkyl copolymer, i.e. dimethicone copolyol, polydimethylsiloxane with stearoxy end groups (stearoxy-dimethicone), polydimethylsiloxane and dialkylammonium acetate copolymer or polydimethylsiloxane and polyalkyl betaine copolymer described in French Patent No. °2 197 352, organo-polysiloxanes modified by mercapto or mercaptoalkyl groups such as those described in French Patent No. FR 1 530 369 and in European Patent Application No. EP 295 780, as well as silanes such as stearoxytrimethylsilane.

The reducing composition according to the present disclosure may also comprise at least one other treating ingredient such as cationic polymers, for instance, those used in the compositions of French Patent Nos. FR 2 472 382 and FR 2 495 931, and for example, cationic polymers such as of the ionene type, or cyclopolymers, basic aminoacids such as lysine or arginine, acidic aminoacids such as glutamic acid or aspartic acid, peptides and their derivatives, protein hydrolyzates or waxes.

The reducing composition according to the present disclosure may also comprise at least one swelling and/or penetrating agent, or at least one agent that can increase the reductor's efficiency, such as a SiO₂/PDMS mixture (polydimethylsiloxane), dimethylisosorbitol, urea and its derivatives, pyrrolidone, N-alkyl-pyrrolidones, alkyleneglycol or dialkyleneglycol alkyl ethers such as, for example, propylene glycol monomethylether, dipropylene glycol monomethylether, ethylene glycol monoethylether and diethylene glycol monoethylether, C₃-C₆ alkanediols such as for example 1,2-propanediol, 1,3-propanediol and 1,2-butanediol, 2-imidazolidinone.

The reducing composition as disclosed herein can also comprise at least one adjuvent, such as fatty alcohols, lanoline derivatives, active agents such as panthothenic acid, agents for combating hair loss, anti-dandruff agents, thickeners, suspension agents, sequestering agents, opacifying agents, dyes, sunscreen agents as well as fragrances and preserving agents.

The reducing composition according to the present disclosure can comprise at least one solvent chosen from water, C₁-C₆ alcohols, for instance alkanols such as ethanol, propanol and isopropanol, alkanediols such as ethylene glycol, propylene glycol and pentanediol, benzyl alcohol, C₂-C₆ ethers, C₂-C₆ esters, N-methylpyrrolidone (NMP) and C₃-C₆ ketones.

The composition can be, for example, in aqueous form. In one embodiment of the present disclosure, for instance, the composition is in the form of a lotion, thickened or not, a cream, thickened or not, or a gel.

Another aspect of the present disclosure is a process for permanent hair reshaping comprising:
applying onto the hair at least one reducing composition as disclosed herein, to reduce the keratin disulfide bonds; and
applying to the hair at least one oxidizing composition, to form the bonds again, or by contacting the hair with atmospheric oxygen.

The at least one photo-oxidizing agent can be, for example, incorporated into the reducing composition when the composition is ready for use. Alternatively, the at least one photo-oxidizing agent can also be applied onto the hair before applying the at least one reducing agent.

Another aspect of the present disclosure is a process for permanently reshaping the hair comprising:
applying to the hair a cosmetic composition comprising, in a cosmetically acceptable medium, at least one photo-oxidizing agent complexed with at least one metal,
applying to the hair a reducing composition comprising, in a cosmetically acceptable medium, at least one reducing agent, and then

applying to the hair at least one oxidizing agent, or contacting the hair with atmospheric oxygen.

The photooxidation reaction of the at least one photo-oxidizing agent can be triggered by exposing hair to a natural or artificial light source.

The hair can be set in form using tools well known to those skilled in the art.

To make a permanent waving, rollers for example, can be used, the reducing composition being applied before or after setting the hair and the oxidizing-fixing composition being applied after the reducing composition, with or without an intermediate or a subsequent step to rinse out or to apply an intermediate composition.

The reducing composition according to the present disclosure can be, for instance, applied to wet hair, which has been previously wrapped around rollers ranging from 2 mm to 30 mm in diameter. The reducing composition can also be applied as hair rolling process progresses. The reducing composition can then be left, for example, for a period of time ranging from 5 minutes to 60 minutes, such as from 15 minutes to 45 minutes, in order to act, then is rinsed out thoroughly. The oxidizing composition can then be applied to wrapped hair, the oxidizing composition enabling the re-formation of the keratin disulfide bonds, and left for a period of time ranging from 2 minutes to 10 minutes. After removing the rollers, the hair is rinsed out thoroughly.

Once the reducing composition has been applied, the hair may also optionally undergo a thermal treatment by heating the hair to a temperature ranging from 30° C. to 60° C., for all or part of the application time. This may be performed using, for example, a helmet hair drier, a hair drier, an infrared radiation drier and other usual heating devices.

A heating iron, the temperature of which ranges from 60° C. to 250° C., such as from 120° C. to 220° C., may also be used both as heating means and as hair setting means, the heating iron being used between the application of the reducing composition and the subsequent oxidation.

To carry out the uncurling or the straightening of the hair, a reducing composition according to the present disclosure is applied onto the hair, then the hair undergoes a mechanical reshaping, so as to get fixed in its new shape, for example, by a smoothing operation with wide toothed comb, with the back of the comb or back of the hand. After a period of time ranging from 5 minutes to 60 minutes, for instance, from 15 minutes to 45 minutes, the hair is smoothed down once again, then carefully rinsed out, and the oxidizing composition is applied such as defined hereinbefore, which is left on for a period of time ranging from about 2 minutes to 10 minutes before rinsing the hair thoroughly.

The oxidizing composition may be any oxidizing composition traditionally used to permanently reshape hair and which comprises at least one oxidizing agent chosen from, for instance, hydrogen peroxide, alkaline bromates, persalts, polythionates and a mixture made of alkaline bromate and persalt. When present in the oxidizing composition, the hydrogen peroxide can be present in an amount ranging from 1 volume to 20 volumes, such as from 1 volume to 10 volumes, relative to the total weight of the oxidizing composition. When present in the oxidizing composition, alkaline bromate can be present in an amount ranging from 2% to 12% by weight, relative to the total weight of the oxidizing composition. When present in the oxidizing composition, persalt can be present in an amount ranging from 0.1% to 15% by weight, relative to the total weight of the oxidizing composition.

The oxidizing composition pH can range from 2 to 10.

As disclosed above, the oxidizing composition may be applied immediately or later, relative to the reducing composition.

According to one embodiment of the the present disclosure, the smoothing of the hair may also be done, in all or part, with a heating iron at a temperature ranging from 60° C. to 250° C., such as from 120° C. to 220° C.

Still another aspect of the present disclosure is a multi-compartment kit for permanently reshaping the hair, comprising, in at least one first compartment, at least one reducing composition as disclosed herein and, in at least one second compartment, at least one oxidizing composition.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Not with standing that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

The following photo-oxidizing compounds were used to formulate reducing compositions according to the present disclosure:
Manganese II phtalocyanine (hereafter referred to as P(Mn))
Iron III phtalocyanine (hereafter referred to as P(Fe))

The following reducing compositions were formulated:

Reducing Composition A1 (free from any photo-oxidizing agent)

| Thioglycolic acid: | 9 | g |
|---|---|---|
| NH₄OH: | to pH 9 | |
| Water: | qs 100 | g |

Reducing Composition A2 (according to the present disclosure)

| Thioglycolic acid: | 9 | g |
|---|---|---|
| P(Mn): | 5 | g |
| NH₄OH: | to pH 9 | |
| Water: | qs 100 | g |

Reducing composition A3 (according to the present disclosure)

| Thioglycolic acid: | 9 | g |
|---|---|---|
| P(Fe): | 5 | g |
| NH₄OH: | to pH 9 | |
| Water: | qs 100 | g |

Fixing composition B

| H₂O₂: | 8 | vol |
|---|---|---|
| Citric acid: | to pH 3 | |
| Water: | qs 100 | g |

The hair was set and shaped using the above defined reducing compositions and fixing composition.

To this end, the hair was washed and towel-dried, then placed under tension with rollers.

One of the Reducing Compositions A1 or A2 or A3 was applied onto the hair and left on for about 15 minutes, at ambient temperature (about 20° C.), under a transparent plastic film that limited the odor developed upon application of the reducing composition.

The plastic film was removed and the odor evaluated.

The hair was rinsed out with water.

Fixing composition B was then applied, and left on the hair for 5 minutes at ambient temperature.

The composition was then rinsed out, rollers were removed and the hair curling was evaluated.

It was noted that for hair treated with Composition A1, the bad odor was much stronger than for hair treated with Compositions A2 or A3, for a similar resulting curling, after hair fixing with composition B.

1. A cosmetic reducing composition for permanently reshaping keratinous fibers, comprising, in a cosmetically acceptable medium, at least one reducing agent and at least one photo-oxidizing agent complexed with at least one metal.

2. The cosmetic reducing composition according to claim 1, wherein the keratinous fibers are hair.

3. The cosmetic reducing composition according to claim 1, wherein the at least one metal is chosen from transition metals.

4. The cosmetic reducing composition according to claim 3, wherein the at least one transition metal is chosen from Ti, V, Cr, Mn, Fe, Co, Ni, Cu, Zn, Mo, Ru, Rh, Pd and Ag.

5. The cosmetic reducing composition according to claim 4, wherein the at least one transition metal is chosen from Cu, Zn, Ni, Fe, Co and Mn.

6. The cosmetic reducing composition according to claim 5, wherein the at least one transition metal is chosen from Fe and Mn.

7. The cosmetic reducing composition according to claim 1, wherein the at least one metal is in a form chosen from metal salts, metal oxides, and metal complexes with at least one ligand.

8. The cosmetic reducing composition according to claim 7, wherein the at least one metal is chosen from metal salts of formula (I):
M(T)ₙ (I)
wherein
M is a metal chosen from Ti, V, Cr, Mn, Fe, Co, Ni, Cu, Zn, Mo, Ru, Rh, Pd and Ag metals,
T is an inorganic anion chosen from sulfate, sulfonate, phosphate, phosphonate, chloride, bromide and nitrate ions; or an organic anion chosen from lactate, citrate and acetate ions; and
n is an integer ranging from 1 to 8.

9. The cosmetic reducing composition according to claim 7, wherein the at least one metal is chosen from metal oxides of formula (II):
M_{m′}(O)ₘ (II)
wherein
M is a metal chosen from Ti, V, Cr, Mn, Fe, Co, Ni, Cu, Zn, Mo, Ru, Rh, Pd, and Ag, m′ is an integer ranging from 1 to 5 and m is an integer ranging from 1 to 8.

10. The cosmetic reducing composition according to claim 7, wherein the at least one metal is chosen from metal complexes with at least one ligand, wherein the complexes are those of formula (III):
M(L)ₚ (III)
wherein
M is a metal chosen from Ti, V, Cr, Mn, Fe, Co, Ni, Cu, Zn, Mo, Ru, Rh, Pd, and Ag,
L is an organic ligand chosen from saturated and unsaturated, cyclic and acyclic compounds, comprising carboxylate and/or primary, secondary or tertiary amine and/or hydroxy groups; or an inorganic ligand chosen from NH₃ and NO groups; and p is an integer ranging from 1 to 10.

11. The cosmetic reducing composition according to claim 10, wherein the at least one ligand is chosen from 1,4,8,11-tetraazacyclotetradecane, 1,8-diazabicyclo[5.4.0]undec-7-ene, ethylenediamine, nitrilotriacetic acid, tris(aminoethyl)amine, tris(aminomethyl)methane, 1,3,5-triaminocyclohexane, dicarboxylic 2,6-pyridine, 2,6-pyridine-dicarboxylic acid, 2,2′,6′,2″-terpyridine, 1,10-phenanthroline, tris(2-pyridylmethyl)amine, tris(2-pyridylethyl)amine and N-carboxymethyl-N-(2-pyridylmethyl)glycine.

12. The cosmetic reducing cosmetic composition according to claim 1, wherein the at least one photo-oxidizing agent is chosen from:
porphyrines,
porphyrine derivatives chosen from chlorine, pheophorbide, pyreophorbide and benzoporphyrine derivatives,
naphtalocyanines,
phtalocyanines; and
cyanocobalamine.

13. The cosmetic reducing cosmetic composition according to claim 12, wherein the at least one phtalocyanine complexed with at least one metal is chosen from the compounds of formula (IV): 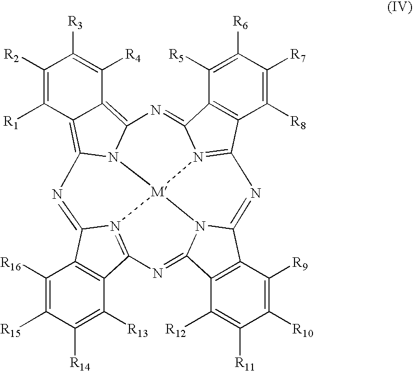
wherein
M′ is a metal chosen from Ti, V, Cr, Mn, Fe, Co, Ni, Cu, Zn, Mo, Ru, Rh, Pd, and Ag, the M′ metal having an oxidation number of greater than zero, and optionally one or more organic or inorganic anions, wherein said inorganic anions are chosen from sulfate, sulfonate, phosphate, phosphonate, chloride, bromide and nitrate ions; and said organic anions are chosen from lactate, citrate and acetate ions; and/or one or more organic or inorganic ligands, wherein said organic ligands are chosen from saturated and unsaturated, cyclic and acyclic compounds, comprising carboxylate and/or primary, secondary or tertiary amine and/or hydroxy groups; and said inorganic ligands are chosen from NH₃ and NO groups;
R₁ to R₁₆, which may be identical or different, are chosen from:
hydrogen,
hydroxy, amino, mono and dialkylamino, mono and dihydroxyalkylamino, alkoxy, halogeno, and cyano groups,
saturated and unsaturated, linear and branched hydrocarbon chains comprising from 1 to 50 carbon atoms, optionally interrupted by at least one entity chosen from heteroatoms, optionally substituted, aromatic and heteroaromatic rings, hydroxy and alkylhydroxy groups, and carbonyl moieties, said chains being optionally substituted by at least one group chosen from hydroxy, amino, alkoxy, halogeno, cyano, mono and dialkylamino, and mono and dihydroxyalkylamino groups,
radicals bearing solubilizing ionic groups, and
radicals bearing at least one reactive groups, which can graft phtalocyanine onto a solid carrier or onto a soluble or insoluble polymer.

14. The cosmetic reducing composition according to claim 13, wherein the at least one transition metal is chosen from Cu, Zn, Ni, Fe, Co and Mn.

15. The cosmetic reducing composition according to claim 14, wherein the at least one transition metal is chosen from Fe and Mn.

16. The cosmetic reducing composition according to claim 13, wherein the at least one heteroatom is chosen from S, O, N, and P atoms.

17. The cosmetic reducing composition according to claim 13, wherein the ionic solubilizing groups are chosen from carboxylate, sulfonate, trialkylammonium, trialkylphosphonium, triarylphosphonium, alkylpyridinium and arylpyridinium groups, alkyl residues ranging from C₁-C₁₈, and aryl residues ranging from C₆-C₁₂.

18. The cosmetic reducing composition according to claim 1, wherein the at least one photo-oxidizing agent is solubilized or dispersed in the composition.

19. The cosmetic reducing composition according to claim 1, wherein the at least one photo-oxidizing agent is adsorbed or grafted onto a solid carrier.

20. The cosmetic reducing composition according to claim 19, wherein the solid carrier is chosen from silica particles, titanium oxide particles, polymer particles and metal particles.

21. The cosmetic reducing composition according to claim 20, wherein the polymer particles are chosen from polypropylene, polyurethane, and styrene-divinylbenzene particles.

22. The cosmetic reducing composition according to claim 1, wherein the at least one photo-oxidizing agent is entrapped within a porous material.

23. The cosmetic reducing composition according to claim 22, wherein the porous material is chosen from polymer particles, three-dimensional lattices resulting from alkoxy silane molecules condensation, and zeolites.

24. The cosmetic reducing composition according to claim 1, wherein the at least one photo-oxidizing agent is present in an amount ranging from 0.1% to 20% by weight, relative to the total weight of the composition.

25. The cosmetic reducing composition according to claim 24, wherein the at least one photo-oxidizing agent is present in an amount ranging from 1% to 10% by weight, relative to the total weight of the composition.

26. The cosmetic reducing composition according to claim 1, wherein the at least one reducing agent is chosen from reducing agents of formula (VI):
(H)_{q}X(R)ᵣ (VI)
wherein X is chosen from P and S atoms, and SO₂ groups, q is 0 or 1, r is 1 or 2 or 3, and R is chosen from linear and branched, saturated and unsaturated (C₁-C₂₀) hydrocarbon radicals, optionally interrupted by at least one heteroatom and optionally substituted with at least one group chosen from hydroxy, halogeno, amine, carboxy, ((C₁-C₃₀) alkoxy)carbonyl, amido, ((C₁-C₃₀)alkyl)amino carbonyl, ((C₁-C₃₀)acyl) amino, mono and di((C₁-C₃₀) alkoxy)carbonyl, and mono and dihydroxy ((C₁-C₃₀)alkyl)amino groups, and salts thereof combined with a base.

27. The cosmetic reducing composition according to claim 1, wherein the at least one reducing agent is chosen from thioglycolic acid, thiolactic acid, glycerol monothioglycolate, cysteamine, N-acetyl-cysteamine, N-propionyl-cysteamine, cysteine, N-acetyl-cysteine, thiomalic acid, pantheteine, 2,3-dimercaptosuccinic acid, N-(mercaptoalkyl)-ω-hydroxyalkylamides, N-mono and N, N-dialkylmercapto-4-butyramides, aminomercapto-alkylamides, N-(mercaptoalkyl)succinamic acid derivatives and N-(mercaptoalkyl)succinimide derivatives, alkylamino mercaptoalkylamides, an azeotropic mixture of 2-hydroxypropyl thioglyconate and (2-hydroxy-1-methyl)ethyl thioglyconate, mercaptoalkylaminoamides, N-mercapto-alkylalkanediamides and formamidine sulfinic acid derivatives and salts thereof.

28. The cosmetic reducing composition according to claim 1, wherein the at least one reducing agent is present in an amount ranging from 0.05% to 30%, relative to the total weight of the composition.

29. The cosmetic reducing composition according to claim 28, wherein the at least one reducing agent is present in an amount ranging from 1% to 20% by weight, relative to the total weight of the composition.

30. The cosmetic reducing composition according to claim 1, further comprising at least one surfactant chosen from alkyl sulfates, alkyl benzene sulfates, alkyl ether sulfates, alkyl sulfonates, quaternary ammonium salts, alkyl betaines, oxyethylene alkyl phenols, fatty acid alkanolamides, oxyethylene fatty acid esters, and hydroxypropylethers.

31. The cosmetic reducing composition according to claim 1, further comprising at least one solvent chosen from water, C₁-C₆ alcohols, alkanediols, benzyl alcohol, C₂-C₆ ethers, C₂-C₆ esters, N-methylpyrrolidone (NMP), and C₃-C₆ ketones.

32. The cosmetic reducing composition according to claim 31, wherein the C₁-C₆ alcohols are alkanols chosen from ethanol, propanol and isopropanol.

33. The cosmetic reducing composition according to claim 31, wherein the alkanediols are chosen from ethylene glycol, propylene glycol and pentanediol.

34. The cosmetic reducing composition according to claim 1, wherein it is in aqueous form.

35. The cosmetic reducing composition according to claim 34, wherein it is in a form chosen from lotions, thickened or not; creams, thickened or not; and gels.

36. A process for permanently reshaping the hair comprising:
applying to the hair at least one reducing composition comprising, in a cosmetically acceptable medium, at least one reducing agent and at least one photo-oxidizing agent complexed with at least one metal, in order to reduce the keratin disulfide bonds, and
applying to the hair at least one oxidizing composition, or contacting the hair with atmospheric oxygen, in order to form disulfide bonds again.

37. The process according to claim 36, wherein the at least one photo-oxidizing agent is incorporated into the reducing composition when said composition is ready for use.

38. A process for permanently reshaping the hair comprising:
applying to the hair at least one cosmetic composition comprising, in a cosmetically acceptable medium, at least one photo-oxidizing agent complexed with at least one metal,
applying to the hair at least one reducing composition comprising, in a cosmetically acceptable medium, at least one reducing agent, and
applying to the hair at least one oxidizing composition or contacting the hair with atmospheric oxygen.

39. The process according to claim 36, wherein the photooxidation reaction of the at least one photo-oxidizing agent is triggered by exposing hair to a natural or artificial light source.

40. The process according to claim 38, wherein the photooxidation reaction of the at least one photo-oxidizing agent is triggered by exposing hair to a natural or artificial light source.

41. The process according to claim 36, wherein the at least one reducing composition is left on for at period of time ranging from 5 minutes to 60 minutes.

42. The process according to claim 41, wherein the at least one reducing composition is left on for at period of time ranging from 15 minutes to 45 minutes.

43. The process according to claim 38, wherein the at least one reducing composition is left on for at period of time ranging from 5 minutes to 60 minutes.

44. The process according to claim 43, wherein the at least one reducing composition is left on for at period of time ranging from 15 minutes to 45 minutes.

45. The process according to claim 36, wherein the at least one oxidizing composition comprises at least one oxidizing agent chosen from hydrogen peroxide, alkaline bromates, persalts, polythionates and a mixture made of alkaline bromate and persalt.

46. The process according to claim 38, wherein the at least one oxidizing composition comprises at least one oxidizing agent chosen from hydrogen peroxide, alkaline bromates, persalts; polythionates and a mixture made of alkaline bromate and persalt.

47. The process according to claim 36, comprising heating the hair with a heating iron with at temperature ranging from 60° C. to 250° C. after applying the reducing composition but before applying the oxidizing composition or air.

48. The process according to claim 47, heating iron temperature ranges from 120° C. to 220° C.

49. The process according to claim 38, comprising heating the hair with a heating iron with at temperature ranging from 60° C. to 250° C. after applying the reducing composition but before applying the oxidizing composition or air.

50. The process according to claim 49, heating iron temperature ranges from 120° C. to 220° C.

51. A multi-compartment kit for permanently reshaping the hair, comprising,
at least one first compartment comprising at least one reducing composition comprising, in a cosmetically acceptable medium, at least one reducing agent and at least one photo-oxidizing agent complexed with at least one metal; and
at least one second compartment comprising at least one oxidizing composition.
